(19) United States
(12) Patent Application Publication
(10) Pub. No.: US 2013/0158525 A1
(43) Pub. Date: Jun. 20, 2013

(54) FLEXIBLE CHANNEL SURGICAL INSTRUMENTS

(71) Applicant: ADN International, LLC, New York, NY (US)

(72) Inventors: Andrew BLITZER, New York, NY (US); Marshall STROME, New York, NY (US)

(73) Assignee: ADN International, LLC, New York, NY (US)

(21) Appl. No.: 13/707,929

(22) Filed: Dec. 7, 2012

Related U.S. Application Data
(60) Provisional application No. 61/569,092, filed on Dec. 9, 2011.

(51) Int. Cl. A61B 17/00

(52) U.S. Cl. CPC A61B 17/00234
USPC 606/1

(57) ABSTRACT

Embodiments described herein are directed to flexible channel instruments for minimally invasive aerodigestive surgical procedures.

### RELATED APPLICATIONS

This application claims the benefit under 35 U.S.C. §119(e) of U.S. provisional application No. 61/569,092, filed Dec. 9, 2011, which is incorporated by reference herein in its entirety.

### FIELD OF THE INVENTION

The embodiments described herein relate to flexible channel instruments for minimally invasive aerodigestive surgical procedures.

### BACKGROUND OF THE INVENTION

Minimally invasive or non-invasive medical procedures of the upper respiratory and/or upper digestive tract are typically performed using endoscopic instruments equipped with channels through which surgical tools are manipulated. A light source and a camera are integrated with the channel and tools such that any movement of the tools results in movement of the light source and visual field. In practice, manipulating surgical tools that are coupled to a light source and/or a camera is cumbersome and problematic.

### SUMMARY OF THE INVENTION

Presented herein are devices and methods for performing minimally invasive surgical procedures in the aerodigestive tract. The inventive devices allow for precise guidance of surgical instruments during particular medical procedures, for example, in the nasal cavity, pharynx, larynx, and esophagus. The devices may have a disposable flexible tip containing channels of variable lengths through which surgical instruments can be manipulated. The flexibility of the tip allows multi-directional movement (e.g., up and down in the vertical plane, left and right in the horizontal plane) and is configured to attach to a handle having a control element for manipulating the flexible tip and/or the surgical instruments within its channels. Typically, the inventive devices are not attached to a light source or a camera. During a surgical procedure, the inventive device is advanced through a subject's nostril, and a separate (independent) light source and/or camera is advanced through the contralateral nostril or mouth to the site of interest in the subject's aerodigestive tract, preferably the upper aerodigestive tract. In this way, a medical professional has the benefit of a fixed visual field while being able to independently manipulate the surgical tools.

Thus, provided herein are devices comprising: a handle having a control element, wherein a portion of the control element is exposed to an end of the handle; and a flexible channel tip, adapted for aerodigestive tract surgery, having at least one working channel for receiving at least one surgical instrument, wherein the flexible channel tip includes at least one guide element disposed therein, wherein the flexible channel tip is detachably connected to the end of the handle such that the guide element interacts with the control element in the handle, and wherein a light source and camera are not attached to the flexible channel tip.

Also provided herein are devices, comprising: a handle having a control element, wherein a portion of the control element is exposed to an end of the handle; and a disposable flexible channel tip, adapted for aerodigestive tract surgery, having at least one working channel for receiving at least one surgical instrument, wherein the disposable flexible channel tip includes at least one guide element disposed therein, wherein the disposable flexible channel tip is detachably connected to the end of the handle such that the guide element interacts with the control element in the handle, and wherein the guide element guides the disposable flexible channel tip in at least four directions.

Further still, provided herein are minimally-invasive surgical methods comprising: advancing an endoscopic light source through a subject's nostril or mouth to a target site located in the subject's nose, nasopharynx, pharynx, or larynx; advancing any one of the devices described herein through the subject's contralateral nostril or mouth to the target site; and manipulating at least one surgical instrument placed through the working channel to perform a surgical procedure.

In some embodiments, the light source and the camera are not attached to the device.

In some embodiments, the handle is made of surgical grade metal, plastic, or a composite.

In some embodiments, the flexible channel tip is disposable.

In some embodiments, the flexible channel tip is made of surgical grade metal, plastic, or a composite.

In some embodiments, the flexible channel tip further comprises at least one spring mechanism.

In some embodiments, the at least one guide element guides the flexible channel tip in at least four directions.

In some embodiments, the at least one surgical instrument is selected from the group consisting of a laser fiber, a cytology brush, an applicator, a needle, forceps, a blade, and a lead.

In some embodiments, the at least one guide element is made of a flexible metal alloy, flexible plastic, silicone, or a combination thereof.

In some embodiments, the flexible channel tip is adapted for surgery of the nasal cavity, pharynx, larynx, trachea, or esophagus.

Also provided herein are kits comprising: a flexible channel tip, adapted for aerodigestive tract surgery, having at least one working channel for receiving at least one surgical instrument, wherein the flexible channel tip includes at least one guide element disposed therein, wherein the flexible channel tip is configured to detachably connect to the end of a handle having a control element, wherein a portion of the control element is exposed to an end of the handle such that the guide element interacts with the control element in the handle, and wherein a light source and camera are not attached to the flexible channel tip; and instructions or direction to obtain instructions for attaching one of the at least one flexible channel tips to the control handle and for use of the handle and flexible tip in an aerodigestive tract surgery.

In some embodiments, the kits also comprise a light source or a camera and/or at least one surgical instrument. The at least one surgical instrument may be selected from the group consisting of a laser fiber, a cytology brush, an applicator, a needle, forceps, and a blade.

In any one of the kits provided herein, the flexible channel tip is disposable, and can be made of surgical grade metal, plastic, or a composite. In some embodiments, the flexible channel tip further comprises at least one spring mechanism.

In any one of the kits provided herein, the at least one guide element guides the disposable flexible channel tip in at least four directions. In some embodiments, the at least one guide element is made of a flexible metal alloy, flexible plastic, silicone, or a combination thereof.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is one embodiment of the inventive device. FIG. 1A depicts a disposable flexible channel tip attached to a non-disposable handle. FIG. 1B depicts a non-disposable handle. FIGS. 1C-1D depict cross-sections of different embodiments of the flexible channel tip.

FIG. 2 is one embodiment of the inventive flexible channel tip.

FIGS. 3A-3F depict several embodiments of a non-disposable handle.

### DESCRIPTION OF SOME EMBODIMENTS OF THE INVENTION

Provided herein are methods and devices for performing minimally invasive surgeries of the aerodigestive tract, including the upper aerodigestive tract. The aerodigestive tract includes for instance, nasopharynx, nose, throat, airway, and esophagus. Aerodigestive surgeries require precise guidance of surgical instruments and simultaneous illumination and visualization of the surgical field. Achieving such precision while maintaining a clear visual field remains a technical challenge. A number of devices have been designed to achieve surgical precision and clarity of view in this area of the body. Nonetheless, the existing devices have many limitations. For example, the endoscopic devices used for surgeries of the upper aerodigestive tract are only capable of moving in two directions along a single plane. These endoscopic devices are attached to a light source such that any manipulation of the device along the single plane results in concordant manipulation of the light source. Thus, not only do the existing devices limit the range of motion that a surgeon can achieve with surgical instruments, but they also limit the extent of illumination/visualization of the surgical area. The devices of the invention provide a solution for minimally-invasive surgeries in this area of the body. For example, having a light source separate from the device provides a steady source of light and allows for adjustment of the light without having to manipulate the device. By providing a device having the structural properties described herein and which is independent of a light source or camera, it is possible to obtain a high level of precision during minimally-invasive surgery of the aerodigestive tract.

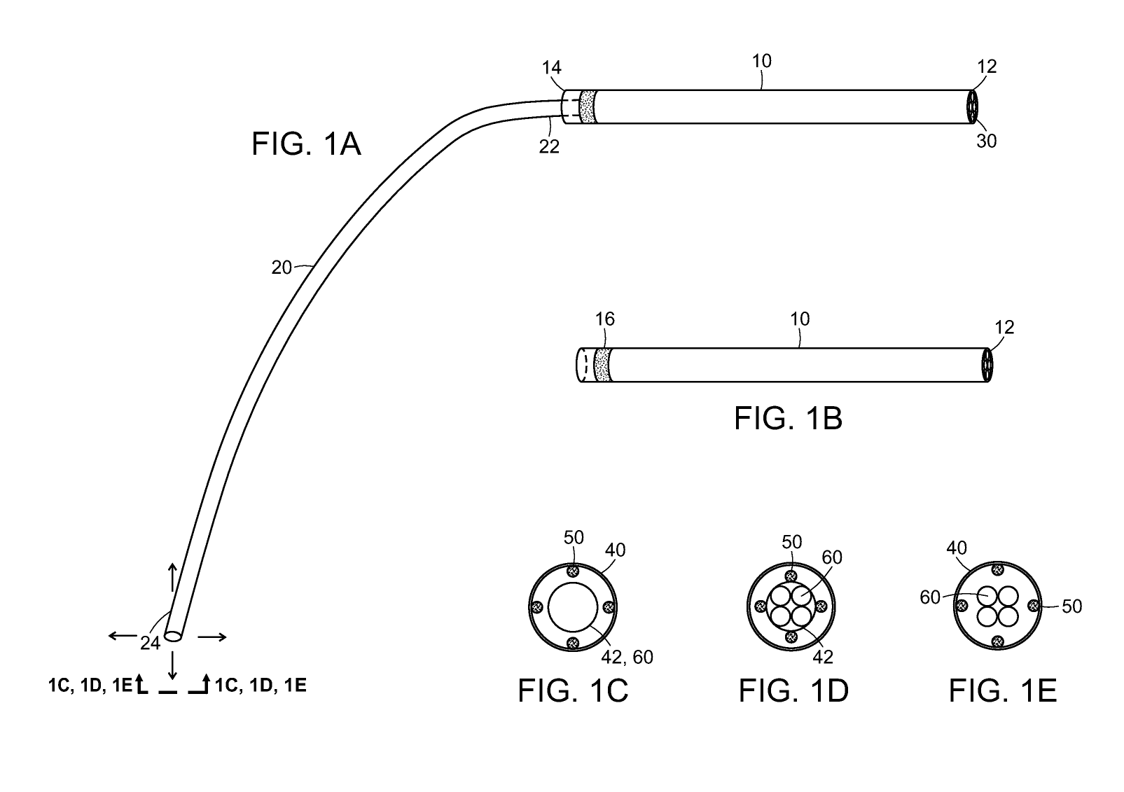

In certain aspects, the devices generally comprise a handle detachably connected to a disposable flexible channel tip that is adapted for use during aerodigestive tract surgery. The flexible channel tip has at least one working channel for receiving at least one surgical instrument therein. In some embodiments, a surgical instrument is disposed within a working channel such that the instrument is protected by the channel from the surrounding environment when the instrument is disposed within the channel and is exposed to the surrounding environment when the instrument is advanced out of the channel. The flexible channel tip also includes at least one guide element disposed therein, which interacts with a control element in the handle such that movement of the flexible channel tip is controlled by manipulation of the handle. In this way, the flexible channel tip can be manipulated in at least four directions (e.g., along the horizontal plane and vertical plane) (see, e.g., FIG. 1A, arrows depicting moving upward, downward, to the left and to the right of the central horizontal axis of the device). Advantageously, a light source and camera are not attached to the flexible channel tip, and in some embodiments, are completely independent of the device itself. An independent endoscopic light source (typically attached to a camera) is advanced through a subject's nostril or mouth to the site of surgery in the subject's nose, nasopharynx, pharynx, or larynx. The surgical instrument(s) is then manipulated through the working channel(s) of the device to perform a surgical procedure, while the light source remains fixed, illuminating the entire surgical area.

FIG. 1A illustrates one embodiment of the device. A handle 10 having a proximal end 12 and a distal end 14 (relative to the person manipulating the device) is detachably connected to the proximal end 22 of a disposable flexible channel tip 20, which can be manipulated in at least four different directions (e.g., up, down, left, right). At least one control element 30 is disposed within the handle 10, and a portion of the control element 30 is exposed to the proximal end 12 of the handle. At least one working channel 60 and at least one guide element 50 is disposed within the flexible channel tip 20 and extends throughout at least the entire length of the tip.

FIGS. 1C-1E depict a cross-sectional view of different embodiments of the flexible channel tip 20. FIG. 1C depicts an inner shaft 42 serving as a working channel 60 and four guide elements 50 attached to the inner surface of the outer shaft 40. FIG. 1C is constructed with one working channel to receive a single surgical instrument, however, the device may be constructed with multiple working channels, each receiving a single surgical instrument. For example, FIG. 1D depicts four working channels 60 disposed within an inner shaft 42 having four guide elements 50 attached to the outer surface of the inner shaft. In some embodiments, the device may be constructed with an inner shaft for additional support. Attachment of the guide elements to the inner surface of the outer shaft, or to the outer surface of the inner shaft, protects the guide elements from the surrounding environment as the device is advanced through the body to the tissue of interest. FIG. 1E depicts four working channels 60 arranged within the flexible channel tip in the absence of a separate inner shaft. In some embodiments, the working channels are constructed to attach to control elements in the handle of the device. In this example, four guide elements 50 are attached to the inner surface of the outer shaft 40.

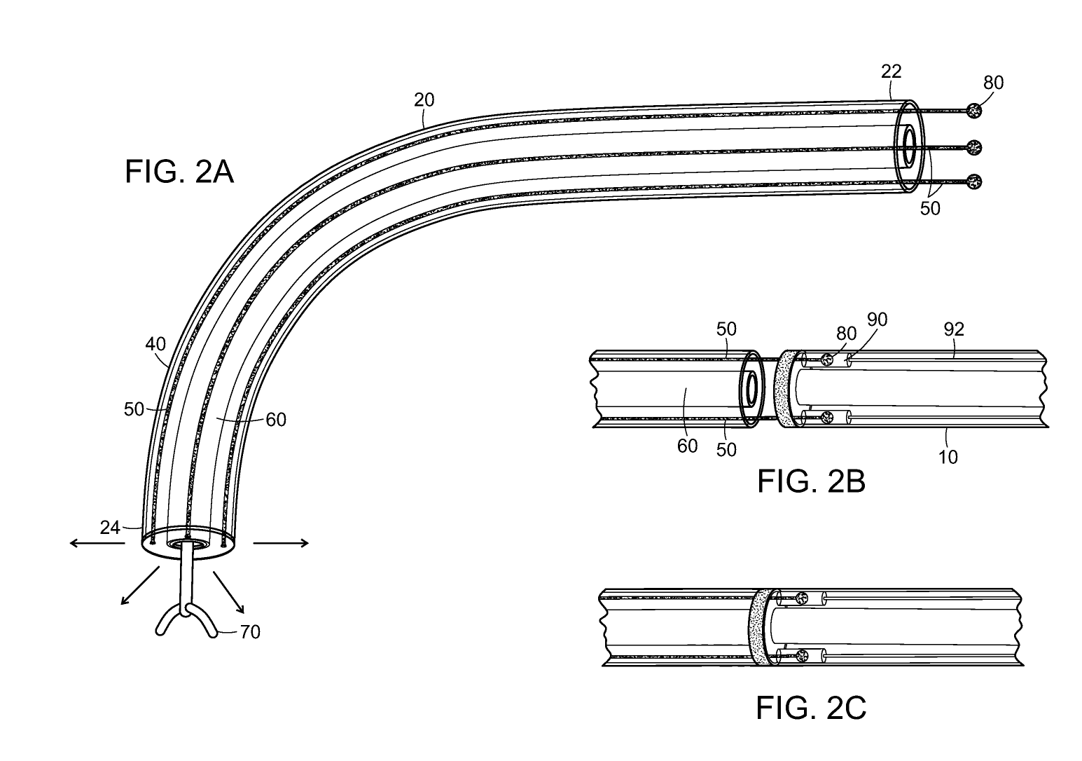

FIG. 2A depicts one embodiment of the flexible channel tip. At least one working channel extends from the proximal end 22 to the distal end 24 of the tip 20. A surgical instrument 70 extends from the distal end of a working channel 60. Guide elements 50 are fixed to the distal end of the flexible channel tip and extend from the proximal end 22 of the tip. In this embodiment, each guide element 50 has a ball-like segment 80 constructed to fit (e.g., lock through an interference fit) into the distal end portion 90 of respective control elements 92 disposed within a handle (FIGS. 2B, 2C). In this way, the guide elements are attached to the control element so that each guide wire can be independently manipulated by a corresponding control element to move the tip, for example, in one of at least four different directions. As an example, by retracting one guide element fixed to the right side of the tip, the tip will pull the surgical instrument such that the tip and the surgical instrument will be deflected to the right. Similarly, by retracting one guide element fixed to the left side of the tip, the tip will pull the surgical instrument to the left.

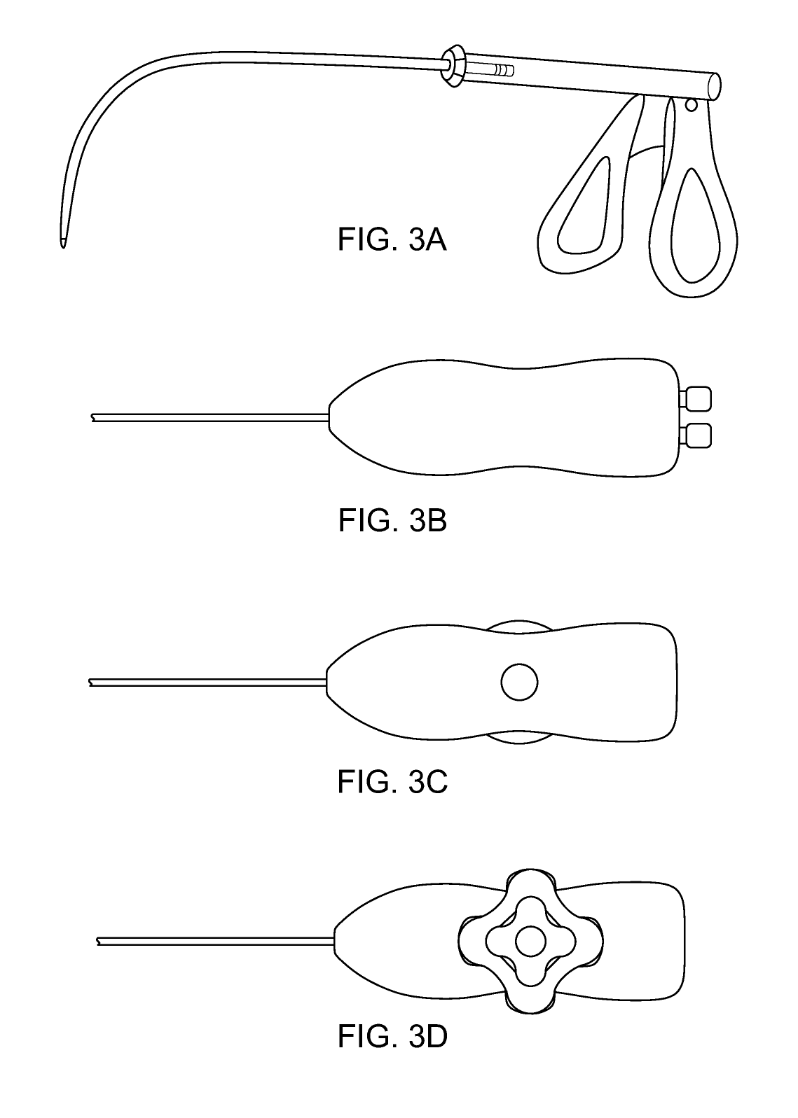

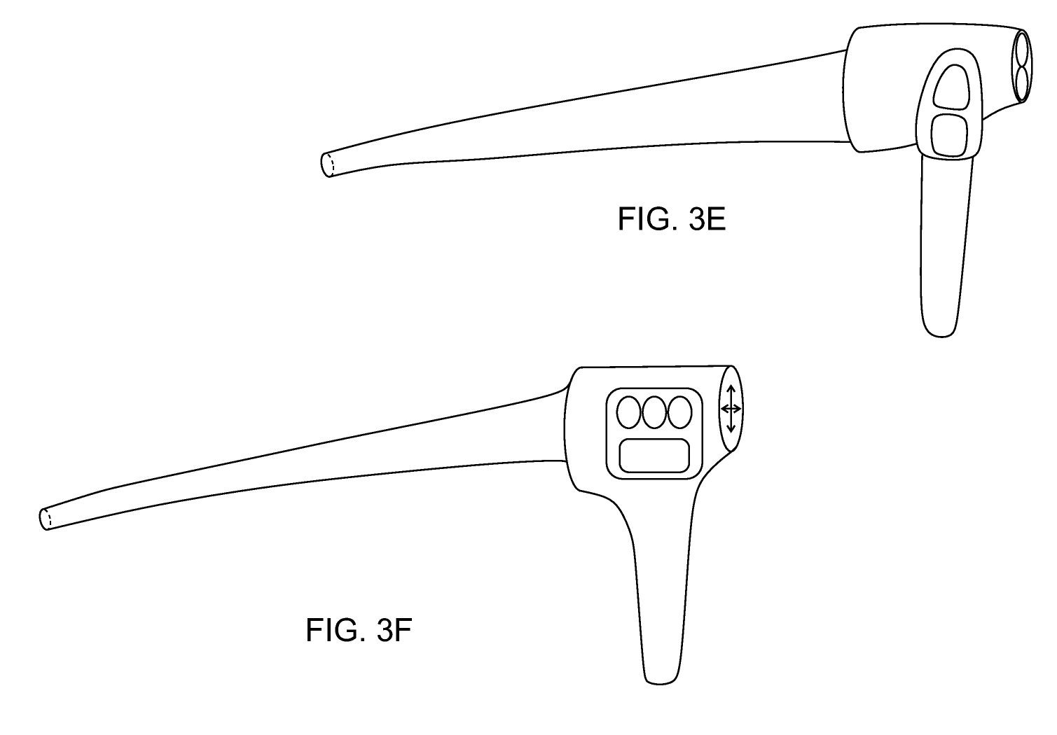

FIGS. 3A-3F depict various embodiments of handles that may be used in combination with the flexible channel tips. The handle may be in a scissor-like configuration (FIG. 3A), contain knobs (FIG. 3B) or various types of discs (FIGS. 3C, 3D) for manually manipulating the control elements and/or working channels of the inventive devices. In some embodiments, for example, when a knob or trigger is actuated, the control element is retracted so as to exert a pulling force on one or more corresponding guide elements, thereby forcing the tip of the device in a particular direction (e.g., up, down, left, right). In some embodiments, when a knob or trigger is actuated, the working channel is extended or retracted. Alternatively, the control elements may be automated such that all is required by, for example a medical practitioner, is a touch of a button that electronically communicates with and controls movement of the control element in the handle, thereby controlling movement of the guide elements and/or working channels (FIGS. 3E, 3F), for example, in one of four directions. Other examples of handles for use with the flexible channel tips are described in U.S. Pat. No. D648,023, incorporated herein by reference.

The flexible channel tip may have a length of between about 100 mm and about 500 mm, depending on the nature of the intended use (see FIG. 2). For example, for minimally invasive surgery of the nasal cavity, a shorter tip (e.g., about 100 mm to about 200 mm) may be used, while a longer tip (e.g., about 250 mm to about 500 mm) may be used for surgery of the esophagus. In some embodiments, the flexible channel tip is about 100 mm, about 150 mm, about 200 mm, about 250 mm, about 300 mm, about 350 mm, about 400 mm, about 450 mm, or about 500 mm in length. The outer diameter of the flexible channel tip (see FIG. 1E) may be between about 10 mm and about 15 mm. In certain embodiments, the outer diameter is about 10 mm, about 11 mm, about 12 mm, about 13 mm, about 14 mm, or about 15 mm. In some embodiments, an inner shaft is sized to fit within the flexible channel tip. The inner shaft may be about 350 mm to about 10 mm in diameter.

In some embodiments, the flexible channel tip is disposable, meaning that it can be discarded after a single use. The tip can be made of any flexible biocompatible material. A plasticizer may be added to any biocompatible material to impart flexibility to that material. Biocompatibility refers to the suitability of a material for exposure to the body or bodily fluids. A material is considered biocompatible if it allows the body to function without complications such as allergic reactions or other adverse side effects. Examples of biocompatible materials for use in making the flexible channel tips include, for example, medical grade polymers such as polyvinyl chloride (PVC), polyethylene, or polypropylene, latex, silicone elastomer, butadiene/acrylonitride copolymers, copolyesters, ethylene vinylacetate (EVB) polymers, ethylene/acrylic copolymers, ethylene/propylene copolymers, polyalkylacrylate polymers, polybutadiene, polybutylene, polyisobutylene, polyisoprene, polyurethane, styrenebutadiene copolymers, and styrene-ethylene/butylene-styrene, polyesters, polyolefins, polyamides, or equivalent or combination thereof. Other materials are well known to the skilled artisan. It is also possible for the flexible tip to be reusable rather than disposable. Such a tip should be able to withstand sterilization techniques, such as heat (e.g., autoclaving) or chemical sterilization.

The flexible channel tip of the device described herein has at least one working channel for receiving a surgical instrument. In some embodiments, the flexible channel tip has an outer shaft 40 and inner shaft 42 (FIGS. 1C, 1D). In some embodiments, the inner shaft 42 is configured to serve as a working channel 60 configured to receive a surgical instrument, and in other embodiments, the inner shaft 42 is configured to receive two or more working channels 60, each channel configured to receive a surgical instrument (FIGS. 1C, 1D). In yet other embodiments, the flexible channel tip has an outer shaft and more than one inner working channels, for example, two, three, or four channels 60, or more, which are not positioned within a separate from an inner shaft (FIG. 1E). In many embodiments, the shaft and/or working channels extend at least the entire length of the flexible channel tip.

The working channels within the flexible channel tip are each configured to receive a surgical instrument. Examples of surgical instruments include fiber optics (e.g., laser fibers), cytology brushes, applicators, needles, forceps, blades, leads, and other surgical instruments used for aerodigestive tract surgeries. In some embodiments, a surgical instrument is detachably connected at the distal end of a working channel. In such embodiments, the proximal end of the channel detachably connects to the distal end of a control element in the handle such that the control element engages with the working channel and/or instrument. This connection permits manipulation of the working channel/instrument via the control element. For example, the control element may be configured to engage with the working channel so as to advance and/or retract the working channel, by any suitable method, through the shaft of the flexible channel tip. Alternatively, or in addition to engagement with the working channel, the control element may be configured to appropriately manipulate the surgical instrument to carry out its function. For example, if the surgical instrument is a set of forceps, actuation of the control element may cause a grasping motion of the forceps to occur. In particular embodiments, there is more than one working channel and instrument. In such embodiments, an individual working channel and/or instrument can be independently advanced or retracted from the tip while the other working channels or instruments temporarily remain tucked inside the tip. Or, an individual instrument may be advanced or retracted relative to a corresponding working channel separately from other instruments.

Various examples of handles that may be used with the devices provided herein are depicted in FIGS. 3A-3F. The device handles may be sized to fit comfortably in the palm of one hand such that the control elements can be manipulated single-handedly, if desired. The handle is typically non-disposable, but in some instances, can be disposable (e.g., single-use). In some embodiments, the handle is made of a medical grade metal, for example, steel. In other embodiments, the handle is made of autoclavable plastic, aluminum, or phenolic. The handle may be custom-configured (ordered to fit) or standardized. Various types of medical device handle shapes are known to those of skill in the art, many of which may be adapted for use with the devices described herein.

The handle comprises at least one control element for manipulating at least one guide element. In certain embodiments, a handle comprises one control element (or set of control elements) that is used to manipulate the guide elements and another control element (or set of control elements) that is used to manipulate the working channels. In many embodiments, the control element is detachably attached to a guide element and/or a working channel. For example, when a disposable flexible channel tip is connected to the handle for use during a surgical procedure, the guide elements and working channels that are housed within the tip are connected to the control elements. On completion of the procedure, the tip, guide elements, and working channels may be disconnected from the handle, and the tip may be discarded.

In some embodiments, the flexible channel tip comprises an interlocking portion at its proximal end and the handle comprises an interlocking portion at its distal end such that when the tip is connected to the handle, the two interlocking portions “lock” together to form a continuous channel. For example, in some embodiments, teeth of one interlocking portion (e.g., of the flexible tip) fit into grooves of an adjacent interlocking portion (e.g., of the handle) such that the interlocking portions do not disconnect from one another when the device is in use. In other embodiments, one interlocking portion is an external thread and the adjacent interlocking portion is an internal thread, much like a nut and bolt mechanism. There are numerous locking mechanisms used to detachably connect two channel-like segments, any one of which may be configured for use with the inventive device.

The flexible channel tip of the device described herein has a guide element, which can be manipulated to move the tip in at least four directions. In some embodiments, the flexible channel tip has two, three, or four guide elements. In particular embodiments, the tip can comprise more than four guide elements. In some embodiments, the guide elements 50 are arranged such that they line the interior periphery of the outer shaft 40 of the flexible channel tip (FIGS. 1C, 1D). In some configurations, the guide elements 50 can be attached directly to the outer surface of the interior shaft 42 (FIG. 1D). In many embodiments, the guide elements extend at least the entire length of the flexible channel tip. Guide elements for use with medical devices are known in the art, and include guide wires, many of which may be configured for use with the inventive device. Examples of guide wires include, but are not limited to: Mirage™, SilverSpeed®, X-Celerator™, and X-Pedion™ guidewires (ev3® Inc., U.S.); ChoICE® Floppy Guide Wire, Lug™ Guide Wire, IQ® Guide Wire, Forte® Floppy Guide Wire (Boston Scientific Corp., U.S.); NiT-Vu™ High-Performance Micro Guidewire, AQUALiner® Hydrophilic Ni—Ti Alloy Guidewire, and PTFE Coated Guidewire (AngioDynamics®, U.S.).

In some embodiments, the flexible channel tip comprises a spring or spring-like mechanism that provides a biasing force. The spring or spring-like mechanism may be located, for example, at the distal end of the tip. Such a spring or spring-like mechanism imparts flexibility to the distal end of the flexible channel tip. In certain embodiments, the distal end of at least one guide element connects to the spring or spring-like mechanism. In some embodiments, tip deflection is controlled by the guide elements via the spring or spring-like mechanism. For example, the spring that controls tip deflection may contribute to the deflection by causing resistance when a force is applied to the working channel.

The flexible channel tip can be deflected in four directions, for example, left and right along a horizontal plane, and up and down along a vertical plane (FIG. 1F). The guide elements disposed within the flexible channel tip are manipulated via a control element in the handle to permit steering of the tip in a desired direction. A “control element” herein refers to any component that controls or steers the movement of a guide element or working channel. An example of a steering mechanism that may be adapted for use with the inventive devices is described in U.S. Pat. No. 5,364,351, incorporated herein by reference. Briefly, to deflect the flexible channel tip in a single direction, a single guide element is retracted while the others (e.g., the second, third, and fourth guide elements) remain static. The steering mechanism may involve at least one rotatable gear and actuatable part, such as a rotatable knob, for manually rotating the gear. A first linearly slidable toothed rack may be attached to the proximal end of each guide element. A toothed gear rotatable by rotation of the knob may engage each of the toothed racks to move them linearly in opposite directions in response to rotation of the gear. In some instances, the guide elements are connected to the racks by connections that transfer tension as the racks move away from the wires but allow the wires to be slack when the rack moves toward its associated wire.

There are many additional steering mechanisms known in the art, for example, those used for steerable catheter systems or those used for steering colonoscopes, any one of which may be adapted for use with the devices provided herein. Examples of various steering mechanisms are detailed in U.S. Pat. Nos. 6,030,360, 5,456,664, 5,454,794, 5,437,636, 5,396,880, 6,872,178, and 7,387,606, each of which is incorporated herein by reference.

With certain methods described herein, a light source is used to illuminate an intended surgical area. In certain embodiments, a light source is an endoscopic light source, such as those provided via fiber optic or LED technology. Non-limiting examples representative of endoscopic light sources that can be used with the embodiments described herein include sinuscopes, rhinoscopes, laryngoscopes, and flexible nasopharyngoscopes (e.g., manufactured by MEDIT INC., Canada). Depending on the medical procedure being performed (e.g., in the nose, throat, or esophagus), in some embodiments, the light source may be about 2 mm to about 10 mm in diameter, and the working length may be about 10 mm to about 500 mm. There are numerous other endoscopic light sources available, and one of skill in the art, for example, a medical practitioner, can easily identify those that are useful with the embodiments provided herein.

With certain methods, a camera is used to visualize/image a surgical procedure. The camera may be attached to or separate from the light source, or the light source may be an integral component of the camera (e.g., not detachable). In some embodiments, the camera is an endoscopic video camera. Non-limiting examples representative of cameras that can be used with the embodiments described herein include those provided by MEDIT INC. (Canada). There are numerous endoscopic cameras available, and one of skill in the art can easily identify those that are useful with the embodiments provided herein. Typically, an endoscopic camera is used with other components, for example, a television monitor or Universal Serial Bus (USB) capture box. A camera/video system may comprise a camera, light source, and video monitor. The camera may be used to pick up the optical images and convert them to an electronic signal that is sent to the video monitor. The light source may comprise a bright light that is focused on the light fiber bundle to transmit light to the distal end of the scope.

A subject, as discussed herein, refers to a human. Preferably the human is a patient in need of minimally-invasive surgery of the aerodigestive tract, preferably the upper aerodigestive tract.

Any one of the foregoing devices and embodiments may be used in a minimally invasive or non-invasive surgical procedure of the aerodigestive tract, preferably the upper aerodigestive tract. The terms “minimally-invasive surgery” and “non-invasive surgery” are used interchangeably herein and refer to any medical procedure which is less invasive than open surgery used for the same purpose. “Open surgery” refers to cutting the skin and tissue to permit direct access to an organ. The minimally- and non-invasive surgeries described herein do not require an incision to access a body organ. Minimally-invasive aerodigestive tract, preferably the upper aerodigestive tract, surgeries include those of the nasal cavity, mouth, pharynx, larynx, (e.g., nasopharynx, oropharynx, hypopharynx, laryngopharynx), trachea, and esophagus.

Nasal cavity and sinus Using the inventive devices, surgical instruments such as biopsy forceps, brush cytology instruments, and laser fibers can be manipulated in the nasal cavity to, for example, cauterize nasal hemorrhage, diminish the size of the nasal turbinates, biopsy growths, vaporize nasal polyps, and sample lesions of the sinuses. The inventive devices can also be used to direct nasal balloon instruments for balloon sinuplasty.

Nasopharynx The inventive devices can be used in the nasopharynx for brush or biopsy sampling of suspected tumors. Laser fibers can also be used to diminish the size of hypertrophic adenoid tissue and to marsupialize cysts. Also contemplated herein is the use of dilators, which can be passed into the Eustachian tube orifice.

Oropharynx and tongue base The inventive devices facilitate biopsy of the oropharynx and tongue base. Laser fibers can be used to shrink lingual or pharyngeal tonsillar tissue. Cysts and mucoceles can be marsupialized using laser fibers and direct endoscopic vision.

Larynx and hypopharynx Biopsy instruments can be used with the inventive devices for sampling of lesions or removal of small lesions in the larynx and hypopharnx. Laser fibers (e.g., those with intense green light lasers) can be used to coagulate vascular lesions or decorticate leukoplakic lesions. Thullium or CO₂ fibers can be used with the inventive devices to vaporize lesions. Other lasers with more specific tissue targets may be used with the devices described herein. Scar bands, small cysts, benign lesions such as papillomas, can all be treated using the inventive devices. In some embodiments, the inventive devices can be used for balloon dilation of the larynx and subglottis.

Esophagus and trachea The upper esophagus and upper trachea may also be treated with balloon dilation, cytology and biopsy procedures using the devices described herein.

As an example, a basic surgical procedure using the inventive devices comprises advancing an endoscope with camera attached (e.g., distal chip camera) through the nose or mouth to a particular area of interest in the aerodigestive tract. The endoscopic camera is then fixed in place, for example, by securing (e.g., by tape/adhesion) the proximal end of the endoscope to the subject's face or other fixed structure. The inventive device is then advanced through the contralateral nostril to the area of interest and is used to manipulate an instrument (e.g., laser fiber, brush, balloon, biopsy forceps) while the visual field remains fixed. Conversely, if the visual field requires readjustment, it can be adjusted without changing the position of the instrument.

In some embodiments, a minimally-invasive esophageal surgery may comprise advancing an endoscopic light source through a subject's nostril to a target site located in the subject's esophagus, advancing one of the devices through the subject's contralateral nostril to the target site, and manipulating a surgical instrument such as a tissue collection brush placed through the working channel to collect a tissue sample from the target site.

In other embodiments, minimally-invasive sinus surgery may comprise advancing an endoscopic light source through a subject's nostril to a target site located in the subject's sinus cavity, advancing one of the devices through the subject's contralateral nostril to the target site, and manipulating a surgical instrument such as a surgical blade placed through the working channel to make one or more incision in the sinus cavity or to remove a small piece of tissue from the cavity. Surgeries of this type may be used to treat, for example, chronic sinusitis, nasal polyps, nasal septal deviations, and blockage of the osteomeatal complex (see e.g., Becker, Daniel, Journal of Long-Term Effects of Medical Implants, 13(3):207-21 (2003), incorporated herein by reference).

In still other embodiments, minimally-invasive laser microsurgery of the mouth and/or throat (trans-oral surgery) may comprise advancing an endoscopic light source through a subject's mouth to a target site located in the subject's mouth or throat, advancing one of the devices through the subject's mouth to the target site, and manipulating a surgical instrument such as a laser placed through the working channel to remove or treat a diseased tissue. Surgeries of this type may be used to treat, for example, a cancer or neoplasm of the mouth or throat.

Each of the foregoing patents, patent applications and references is hereby incorporated by reference.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

We claim:

1. A device, comprising:
a handle having a control element, wherein a portion of the control element is exposed to an end of the handle; and
a flexible channel tip, adapted for aerodigestive tract surgery, having at least one working channel for receiving at least one surgical instrument, wherein the flexible channel tip includes at least one guide element disposed therein, and wherein the flexible channel tip is detachably connected to the end of the handle such that the guide element interacts with the control element of the handle, and wherein a light source and camera are not attached to the flexible channel tip.

2. The device of claim 1, wherein the light source and the camera are not attached to the device.

3. The device of claim 1, wherein the handle is made of surgical grade metal, plastic, or a composite.

4. The device of claim 1, wherein the flexible channel tip is disposable.

5. The device of claim 1, wherein the flexible channel tip is made of surgical grade metal, plastic, or a composite.

6. The device of claim 1, wherein the flexible channel tip further comprises at least one spring mechanism.

7. The device of claim 1, wherein the at least one guide element guides the flexible channel tip in at least four directions.

8. The device of claim 1, wherein the at least one surgical instrument is selected from the group consisting of a laser fiber, a cytology brush, an applicator, a needle, forceps, a blade, and a lead.

9. The device of claim 1, wherein the at least one guide element is made of a flexible metal alloy, flexible plastic, silicone, or a combination thereof.

10. The device of claim 1, wherein the flexible channel tip is adapted for surgery of the nasal cavity, pharynx, larynx, trachea, or esophagus.

11. A device, comprising:
a handle having a control element, wherein a portion of the control element is exposed to an end of the handle; and
a disposable flexible channel tip, adapted for aerodigestive tract surgery, having at least one working channel for receiving at least one surgical instrument, wherein the disposable flexible channel tip includes at least one guide element disposed therein, and wherein the disposable flexible channel tip is detachably connected to the end of the handle such that the guide element interacts with the control element of the handle and wherein the guide element is configured to guide the disposable flexible channel tip in at least four independent directions.

12. The device of claim 11, wherein a light source and a camera are not attached to the device.

13. The device of claim 11, wherein the handle is made of surgical grade metal, plastic, or a composite.

14. The device of claim 11, wherein the disposable flexible channel tip is made of surgical grade metal, plastic, or a composite.

15. The device of claim 11, wherein the disposable flexible channel tip further comprises at least one spring mechanism.

16. The device of claim 11, wherein the at least one surgical instrument is selected from the group consisting of a laser fiber, a cytology brush, an applicator, a needle, forceps, a blade, and a lead.

17. The device of claim 11, wherein the at least one guide element is made of a flexible metal alloy, flexible plastic, silicone, or a combination thereof.

18. The device of claim 11, wherein the flexible channel tip is adapted for surgery of the nasal cavity, pharynx, larynx, trachea, or esophagus.

19. A non-invasive surgical method comprising:
advancing an endoscopic light source through a subject's nostril or mouth to a target site located proximate to the subject's nose, nasopharynx, pharynx, or larynx;
advancing the device of claim 1 through the subject's contralateral nostril or mouth to the target site; and
manipulating at least one surgical instrument disposed in a space defined by the working channel to perform a surgical procedure.

20. A kit comprising:
a flexible channel tip, adapted for aerodigestive tract surgery, having at least one working channel for receiving at least one surgical instrument, wherein the flexible channel tip includes at least one guide element disposed therein, wherein the flexible channel tip is configured to detachably connect to the end of a handle having a control element, wherein a portion of the control element is exposed to an end of the handle such that the guide element interacts with the control element in the handle, and wherein a light source and camera are not attached to the flexible channel tip; and
instructions or direction to obtain instructions for attaching one of the at least one flexible channel tips to the control handle and for use of the handle and flexible tip in an aerodigestive tract surgery.

21-29. (canceled)
